(19) United States
(12) Patent Application Publication
(10) Pub. No.: US 2004/0023872 A1
(43) Pub. Date: Feb. 5, 2004

(54) Use of gadd34 or pcna polypeptides in the treatment of cerebral ischaemic damage

(76) Inventors: Suzanne Moira Brown, Glasgow (GB); Iseabail Mhairi Macrae, Glasgow (GB); James McCulloch, Edinburgh (GB); June Elizabeth Harland, Glasgow (GB)

Correspondence Address: MYERS BIGEL SIBLEY & SAJOVEC, PO BOX 37428, RALEIGH, NC, 27627, US

(21) Appl. No.: 10/344,466

(22) PCT Filed: Aug. 16, 2001
(86) PCT No.: PCT/GB01/03673

(30) Foreign Application Priority Data
Aug. 16, 2000 (GB) 0020067.5

(51) Int. Cl. A61K 48/00; A61K 38/17

(52) U.S. Cl. 514/12; 514/44

(57) ABSTRACT

The localisation of GADD34 and PCNA polypeptides and antibodies in regions of cerebral ischaemic damage is described. The use of such polypeptides, and homologues and derivatives thereof, for treatment or diagnosis of such damage is also described. The polypeptides may be used for preparation of medicaments for treatment of ischaemic damage, or in the diagnosis of such damage,

The present invention relates to the identification of compounds active during cerebral ischaemia, and to the use of such compounds in treating or preventing cerebral ischaemic damage. The invention further relates to antibodies to such compounds, and to their use in diagnosis or treatment of cerebral ischaemic damage.

The importance of cerebral ischaemia is a consequence of the prevalence of cerebrovascular disease and the severity of its sequelae. Cerebrovascular disease ranks third as a cause of death in Western countries, after cancer and heart disease. Ischaemic damage to the brain is a feature of a number of clinical conditions, most notably head injury and stroke.

Reductions in cerebral blood flow, whether through stroke or head injury, initiate multiple neurochemical cascades which lead over time to irreversible damage to brain tissue. Pharmacological intervention directed at these cascades may have the effect of protecting brain tissue from damage. However, while strong evidence of the. therapeutic potential of these interventions has been found in animal models, the results of clinical trials in human head injury and stroke have been less definitive. The only current effective treatment for stroke is tPA (tissue plasminogen activator) which, for reasons of safety and logistics, is available to less than 1% of the stroke population.

An alternative treatment strategy is suggested by the observation that subjection of brain tissue to trauma such as reduction in cerebral blood flow, like subjection of many other cell types to trauma, leads to the activation of multiple genes which promote either the survival or programmed cell death of the brain tissue, particularly at the margins or penumbra of the ischaemic lesion. However, before now neither the exact genes involved nor effective strategies for intervention have been identified.

Use has now been made of a rat model of cerebral ischaemia to identify two genes which are expressed during ischaemia, and to study the development of the ischaemic lesion over time.

Experimental stroke can be induced in the rat to produce a reproducible and quantifiable volume of irreversibly damaged brain tissue (infarct). Experimental stroke can be produced by diathermy occlusion of the middle cerebral artery via a sub-temporal craniectomy. Alternatively, introduction of an intraluminal filament into the carotid artery and advancement of the filament towards the circle of Willis blocks the origin of the middle cerebral artery and produces reproducible ischaemia in the cortex and caudate nucleus. Reperfusion of blood into this region can be achieved by withdrawal of the filament. Ischaemia of 2 hours duration followed by 22 hours of reperfusion results in substantial irreversible damage to regions of the cortex and caudate nucleus. This volume of infarct is not maximal at 24 hours, and further tissue in the peri-infarct (penumbral) zone will be recruited into the infarct over the next 24 hours. Previous studies indicate that between 48 and 72 hours the infarct does not increase further in size. Therefore at 24 hours there is a region of salvageable tissue which, if no intervention occurs, will become irreversibly damaged over the following 24 hours.

Immunohistochemical studies of the rat ischaemia model have indicated the presence of two proteins in the ischaemic territory, specifically GADD34 and PCNA.

GADD34 (Growth Arrest and DNA Damage) (Fornace et al., 1989) is a cellular protein which is thought to have a role in blocking growth and DNA replication following damage, and thus may act as a tumour suppressor gene. The amino terminal domain of GADD34 has been implicated in an apoptopic pathway, while the carboxy terminal domain has been shown to share homology with the mouse myeloid differentiation protein MyD116 (Lord et al., 1990) and the herpes simplex virus (HSV) protein ICP34.5 (McGeoch and Barnett, 1991). One function of the ICP34.5 protein seems to be in preventing host cell protein synthesis shut-off (Chou & Roizman 1992; He et al. 1997;Novoa et al. 2001), so enabling an HSV-infected cell to survive and replicate the HSV.

The conserved carboxy domain of these proteins is known to have a binding affinity with the second identified protein, PCNA (Proliferating Cell Nuclear Antigen), which is itself thought to be involved in cell cycle control and DNA repair (Prelich et al., 1987).

It is thought that GADD34 and MyD116 interact with PCNA to affect cell cycle regulation, while ICP34.5 interacts with PCNA to allow cellular and hence HSV DNA replication to proceed. The function and characteristics of these proteins, and the identification of a human homologue of GADD34, are described in more detail in International Patent Application WO98/41873, the contents of which are incorporated herein by reference.

The present invention relies on the identification and localisation of the GADD34 and PCNA proteins in a cerebral ischaemic lesion.

Using the rat ischaemia models described above, it has been identified that there is upregulation of GADD34 in the brain, specifically in the penumbra of the stroke lesion. Strong, specific immunohistochemical staining is found within cells with low background staining of the neuropil. Immunopositive cells are present in the peri-infarct region of the cortex, caudate nucleus, and in the sub-cortical white matter tract.

There is no detectable expression of GADD34 in the core of the lesion where total cell death has occurred (the infarct), and there is no detectable expression of GADD34 in the normal regions of the brain such as the contralateral hemisphere.

PCNA is also specifically upregulated in the same region as GADD34, with virtually no detectable PCNA in the normal brain or in the infarct.

In the region of upregulation, it is likely that there are cells in which both proteins are expressed, and there are cells in which only one of the proteins is expressed.

It appears that these proteins play a role in the growth of the infarct after the initial insult, and that the cells in which one or both of the proteins are expressed are either trying to recover from the ischaemic insult and are in the process of regeneration; or are in the process of dying. It is likely that there will be separate populations of cells in which each of these processes are taking place. Therefore, intervention and treatment with appropriate compounds could affect the growth of the infarct. It is believed also that similar processes occur in the brain after other forms of pathology; for example, acute insults, physical damage, and neurodegenerative diseases. The present invention may therefore also be of use in diagnosis and treatment of such traumas.

According to a first aspect of the present invention, therefore, there is provided the use of a GADD34 polypeptide, or a homologue or a derivative thereof, in the preparation of a medicament for the treatment of cerebral ischaemic damage. Introduction of excess GADD34 to a cell on the penumbra of the infarct may aid the cell in its recovery from the ischaemic insult, and so restrict the growth of the inf arct. Alternatively, derivatives of the GADD34 polypeptide may be used to modulate activity or the effects of native GADD34 in damaged cells, if this is desired. Polynucleotide derivatives of the GADD34 polypeptide may be used to modulate expression of GADD34.

Homologues of GADD34 include human homologues as described in WO98/41873, MyD116 or ICP34.5. Homologues also include other polypeptide sequences with at least 70%, preferably 80%, more preferably 90%, and most preferably 95% amino acid homology (identity) with the polypeptide sequence or a substantial region thereof of GADD34.

Derivatives of GADD34 include fragments of the complete GADD34 peptide sequence and homologues thereof; and modified compounds derived from the peptide, for example, by glycosylation, amidation, carboxylation, phosphorylation and the like. Derivatives of the GADD34 polypeptide also include polynucleotide sequences selected or designed to express such a polypeptide when introduced to the appropriate enzymatic machinery.

The medicament may comprise a pharmaceutically active compound derived from GADD34; or may include viral vectors (for example, HSV) designed to introduce a DNA or RNA fragment encoding a GADD34 polypeptide into a brain cell; unmodified GADD34; “naked” polynucleotide fragments; antibodies or active fragments thereof raised against epitopes of the GADD34 polypeptide; antisense polynucleotide fragments designed against the genomic DNA encoding the GADD34 polypeptide; and the like.

The medicament may also be provided in conjunction with an appropriate delivery medium and/or device: for example, lipid encapsulation of a polypeptide; pharmacologically acceptable carriers; viral particles; injectable devices; and the like.

Where the medicament includes a polynucleotide fragment intended to be expressed in vivo, the fragment may also be provided in conjunction with appropriate nucleotide promoter, regulator, and targeting sequences. For example, as the infarct region is likely to be hypoxic, it may be convenient to couple the polynucleotide fragment to a hypoxic response element (HRE) promoter, so that the peptide is only expressed in the infarct region.

According to a further aspect of the present invention, there is provided the use of a PCNA polypeptide, or a homologue or a derivative thereof, in the preparation of a medicament for the treatment of cerebral ischaemic damage. This aspect of the invention may be of similar utility to the use of a GADD34 polypeptide, homologue, or derivative described above.

According to a further aspect of the present invention, there is provided a pharmaceutical formulation comprising GADD34 or a physiologically acceptable salt, ester, or other physiologically functional derivatives thereof and a carrier therefor for use in treatment of cerebral ischaemic damage in a human or other animal.

According to a yet further aspect of the present invention, there is provided a pharmaceutical formulation comprising PCNA or a physiologically acceptable salt, ester, or other physiologically functional derivatives thereof and a carrier therefor for use in treatment of cerebral ischaemic damage in a human or other animal.

Examples of physiologically acceptable salts of GADD34 or PCNA include acid addition salts formed with organic carboxylic acids such as acetic, lactic, tartaric, maleic, citric, pyruvic, oxalic, fumaric, oxaloacetic, isethionic, lactobionic. and succinic acids; organic sulfonic acids such as methanesulfonic, ethanesulphonic, benezenesulfonic and p-tolunesulphonic acids and inorganic acids such as hydrochloric, sulphuric, phosphoric and sulphamic acids.

Physiologically functional derivatives of GADD34 or PCNA are derivatives which can be converted in the body into the parent compound, or be active in their own right. Such physiologically functional derivatives may also be referred to as “pro-drugs” or “bioprecursors”.

GADD34 or PCNA or a physiologically acceptable salt, ester or other physiologically functional derivative. thereof may be administered alone or in combination with other drugs as part of a therapeutic regimen for the treatment of cerebral ischaemic damage, or it may be administered as an adjunct to other forms of therapy.

The amount of a compound of GADD34 or PCNA required for use in the treatment of cerebral ischaemic damage will depend inter alia on the route of administration, the age and weight of the patient and the nature and severity of the condition being treated and will ultimately be at the discretion of the attendant physician or veterinarian. In general, a suitable dose for administration to man is in the range of 0.1 to 100 mg. per kilogram bodyweight per day, for example from 1 mg/kg. to 40 mg/kg., per day particularly 5 to 15 mg/kg. per day. For administration by inhalation the dose may conveniently be in the range of 0.1 to 50 mg/kg/day, eg. 1 to 10 mg/kg/day.

It will be appreciated that for administration to neonates, lower doses may be required.

For use according to the present invention GADD34 or is PCNA is preferably presented as a pharmaceutical formulation, comprising GADD34 or PCNA, respectively, or a physiologically acceptable salt, ester or other physiologically functional derivative thereof (hereinafter referred to as “active compound”) together with one or more pharmaceutically acceptable carriers therefor and optionally other therapeutic and/or prophylactic ingredients. The carrier(s) must be acceptable in the sense of being compatible with the other ingredients of the formulation and not deleterious to the recipient thereof.

An active compound may conveniently be presented as a pharmaceutical formulation in unit dosage form. Convenient unit dose formulation contains an active compound in an amount of from 25 mg to 100 mg.

Pharmaceutical formulations include those suitable for oral, topical (including dermal, buccal and sublingual), rectal or parenteral (including subcutaneous, intradermal, intramuscular and intravenous), nasal and pulmonary administration eg. by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of an active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tables may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed for example in a rice paper envelope. An active compound may also be formulated as dispersable granules, which may for example be suspended in water before administration, or sprinkled on food. The granules may be packaged eg. in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms eg. tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles. Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation eg. subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range 0.5 to 7 microns are delivered into the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomiser or nebuliser whereby an accelerated airstream or ultrasonic agitation is employed to produce a fine droplet mist for inhalation.

Formulations suitable for nasal administration include presentations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, as appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w, and preferably 60 to 80% w/w of the active ingredient(s), and 0 to 50% w/w and preferably 20 to 40% w/w of conventional veterinary excipients. These powders may either be added to animal feedstuffs, for example by way of an intermediate premix, or diluted in animal drinking water.

As it is known that GADD34 has a binding affinity for PCNA, it may be that the interaction between the two peptides has some effect on the progress of cerebral ischaemic damage. International Patent Application WO98/41873 describes methods for identifying substances capable of disrupting the interaction between GADD34 and PCNA.

According to a further aspect of the present invention, there is provided the use of a substance capable of disrupting an interaction between a GADD34 polypeptide, or a homologue or a derivative thereof, and a PCNA polypeptide, or a homologue or a derivative thereof, in the preparation of a medicament for the treatment of cerebral ischaemic damage.

According to a yet further aspect of the present invention, there is provided a pharmaceutical formulation comprising a substance capable of disrupting an interaction between a GADD34 polypeptide and a PCNA polypeptide, or a physiologically acceptable salt, ester, or other physiologically functional derivatives thereof and a carrier therefor for use in treatment of cerebral ischaemic damage in a human or other animal.

According to a still further aspect of the present invention, there is provided a method of treatment of cerebral ischaemic damage, the method comprising the step of administering a pharmacologically active dose of a GADD34 polypeptide, or a homologue or a derivative thereof, to a human or animal patient in need of such treatment.

According to a yet further aspect of the present invention, there is provided a method of treatment of cerebral ischaemic damage, the method comprising the step of administering a pharmacologically active dose of a PCNA polypeptide, or a homologue or a derivative thereof, to a human or animal patient in need of such treatment.

According to a further aspect of the present invention, there is provided a method of treatment of cerebral ischaemic damage, the method comprising the step of administering a pharmacologically active dose of a substance capable of disrupting an interaction between a GADD34 polypeptide, or a homologue or a derivative thereof, and a PCNA polypeptide, or a homologue or a derivative thereof, to a human or animal patient in need of such treatment.

Since it has been found that antibodies to GADD34 highlight the penumbra of the lesion, such antibodies could be used for targeted delivery of drugs or other compounds to the penumbra of the lesion.

Thus, according to a further aspect of the present invention, there is provided the use of an antibody to a GADD34 polypeptide for the delivery of a drug for treatment of cerebral ischaemic damage. The term “antibody” as used herein is intended to include polyclonal or monoclonal antibodies or fragments thereof which are specifically reactive with GADD34 polypeptides or immunogenic fragments thereof. Antibodies can be fragmented and the fragments screened for utility using known techniques. For example, F(ab′)₂ fragments can be generated by treating antibody with pepsin. The resulting F(ab′)₂ fragment can be treated to reduce disulphide bridges to produce Fab′ fragments. The antibodies described herein are further intended to include bispecific and chimaeric molecules possessing an antigenic determinant to GADD34.

The present invention yet further provides a method of diagnosis of cerebral ischaemic damage, the method comprising the steps of administering antibodies to a GADD34 polypeptide to a patient; and detecting the region of localisation of said antibodies. The method thus enables a physician to determine the extent of damage in the early stages of ischaemia. The method may also be used for research, for example, detecting the extent of a lesion in a rat model.

The antibodies may be labelled in some way (for example, fluorescently or radioactively labelled, or with an enzyme cleavable substrate, the product of which may be detected); or may be detected by secondary means (for example, by introducing labelled antibodies to the GADD34 antibodies to the subject).

These and other aspects of the present invention will now be described by way of example only, and with reference to the accompanying figures, which show:

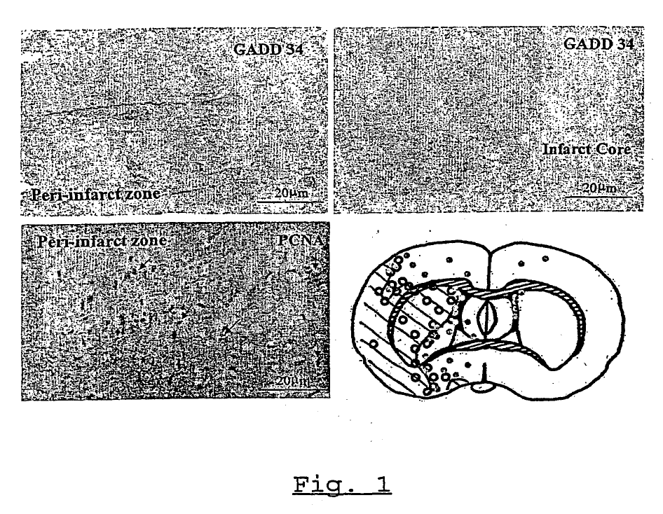

FIG. 1. GADD34 and PCNA immunohistochemistry in the 2 hour MCA occlusion group. GADD34 positive cells were predominantly found in the cortical peri-infarct zone (top left). Minimal GADD34 immunostaining was present in the contralateral (non-ischaemic) hemisphere (top right). No PCNA positive cells were detected in the ipsilateral cortex and caudate but were present bilaterally in the subventricular zone lining the ventricles (bottom left). The diagrammatic representation (bottom right) displays the pattern of GADD34 (open circles) and PCNA (closed circles) positive cells in relation to ischaemic damage (shaded area) at a coronal level in the core of MCA territory. PCNA immunopositive cells in the subventricular zone were also immunopositive for GADD34.

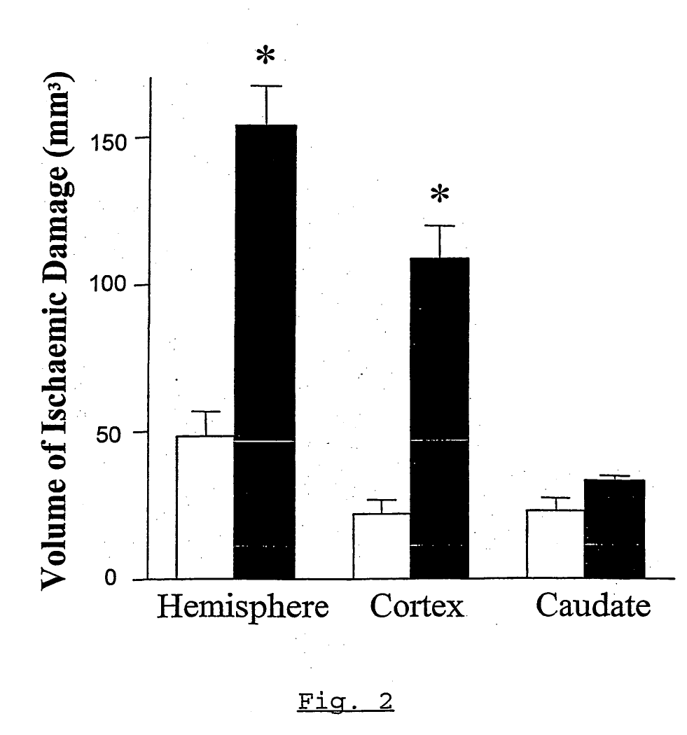

FIG. 2. Volume of neuronal perikaryal damage at 2 hours (open bars; n=6) and 24 hours (solid bars; n=5) after MCA occlusion. Data are mean±S.E.M.

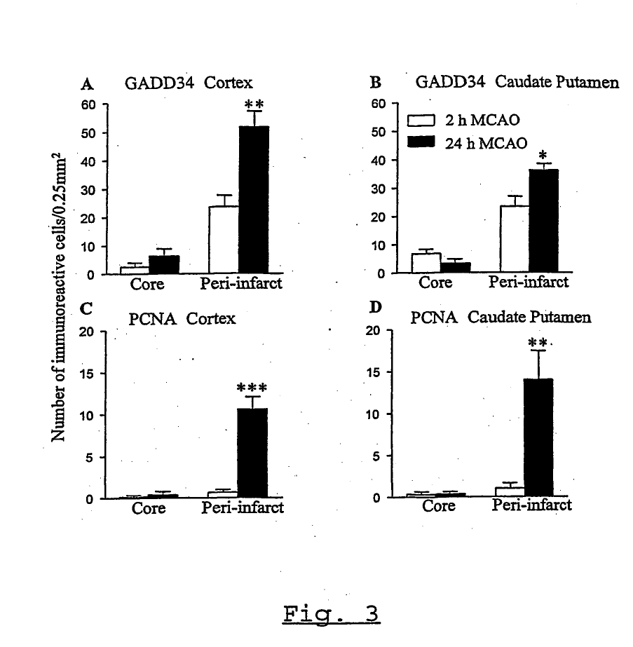

FIG. 3. Quantitative data for GADD34 and PCNA immunopositive cells at 2 (open bars; n=6) and 24 (solid bars; n=5) hours after MCA occlusion in the ipsilateral hemisphere. Cells were counted microscopically using a 100 mm² graticule at ×200 magnification and expressed as the number of cells per 0.25 mm². Data are mean±S.E.M. Apart from the subependymal cells lining the ventricles, immunopositive cells were less frequently detected in the contralateral hemisphere.

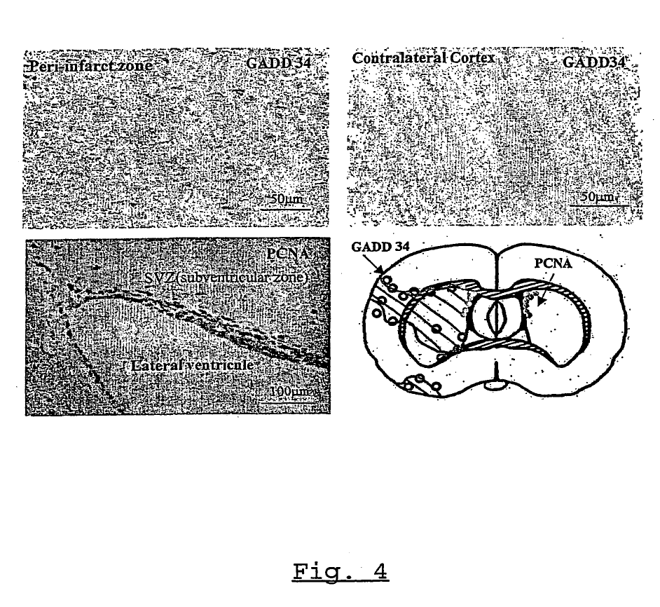

FIG. 4. GADD34 and PCNA immunohistochemistry in the 24 hour MCA occlusion group. GADD34 positive cells were predominantly found in the peri-infarct zone in the cortex (top left) and to a lesser extent in the ischaemic core (top right). PCNA positive cells were detected in the peri-infarct zone (bottom left) and also extended out to the cingulate cortex of the ipsilateral hemisphere. Scattered PCNA positive cells were also present in the contralateral cingulate cortex and lining the subventricular zone bilaterally., The diagrammatic representation (bottom right) displays the pattern of GADD34 (open circles) and PCNA (solid circles) positive cells in relation to ischaemic damage, (shaded area) at a coronal level in the core of MCA territory.

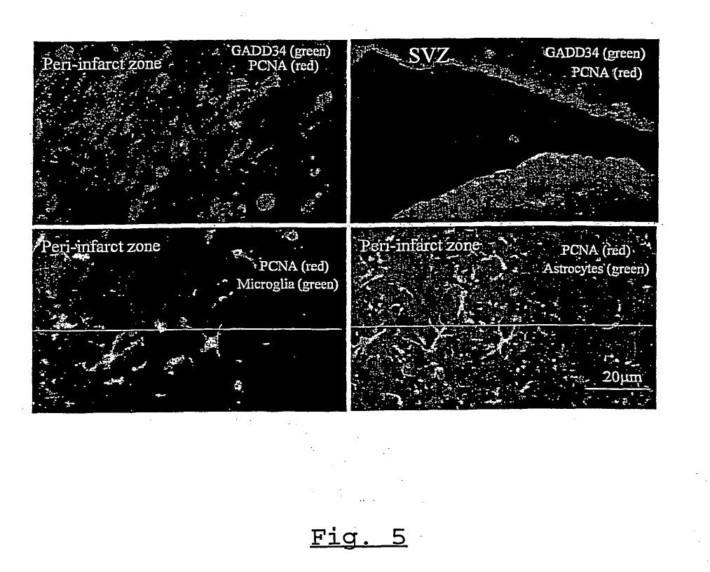

FIG. 5. Scanning confocal laser microscopy of the per-infarct zone at 24 hours. Top row: most neurones (Neu-N) in the field are GADD34 positive. Bottom row: no astrocytes in the field (GFAP) are GADD34 positive.

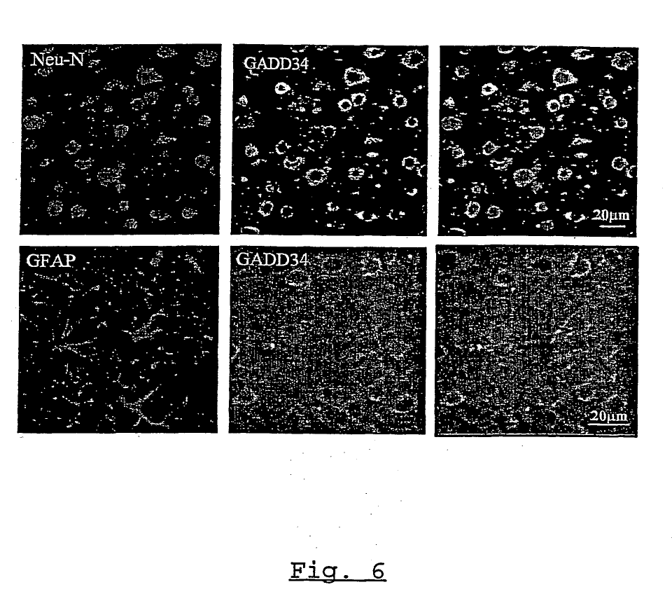

FIG. 6. Double-labelling fluorescence photomicrographs from the ipsilateral cortex peri-infarct zone and subventricular zone (SVZ) 24 hours after MCA occlusion. In the peri-infarct zone (top left) cells predominantly display increased GADD34 immunoreactivity and a number of these cells are also PCNA-positive; subependymal cells of subventricular zone (top right) displayed strong PCNA immunoreactivity in nucleus. Basement cytoplasm of the ependymal layer also displays GADD34 immunoreactivity. The vast majority of PCNA positive cells in the peri-infarct zone (bottom left). co-localize with Mrf-1 identifying them as microglia. Some PCNA immunopositive cells in the field are not Mrf-1 positive. Astrocytes (bottom right) displaying strong GFAP immunoreactivity showed no co-localization with PCNA (red).

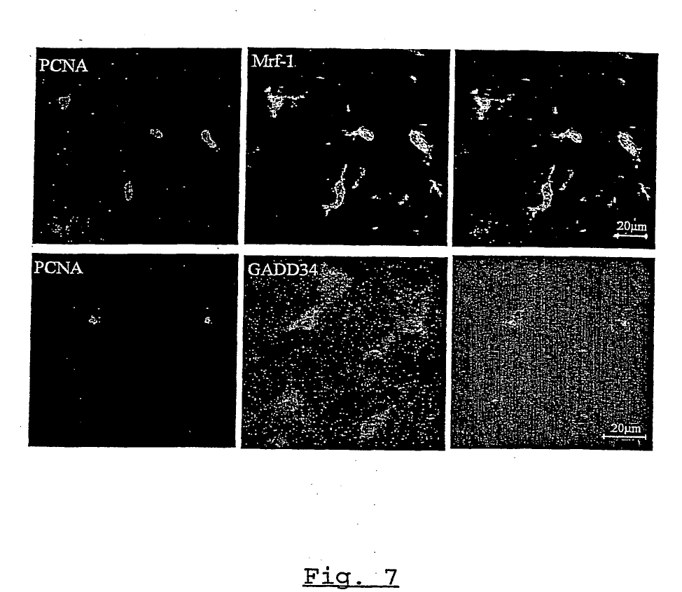

FIG. 7. Scanning confocal laser microscopy of per-infarct zone at 24 hours. Top row: PCNA-positive cells are identified as microglia (Mrf-1 positive). Bottom row: PCNA-positive cells are GADD34-positive but some GADD34 positive cells are PCNA negative.

### MATERIALS AND METHODS

All experimental procedures were carried out under an appropriate Home Office Licence and regulations specified in the Animals (Scientific Procedures) Act 1986. Male, Sprague Dawley rats (weight 250-350 g, Harlan Olac, Bicester, UK) used in the present study were kept in a 12 hour light/dark regime with constant access to food and water.

Induction of Focal Cerebral Ischaemia

Occlusion of the middle cerebral artery (MCA) was carried out by electrocoagulation (diathermy occlusion) of the MCA or by insertion of an intraluminal thread into the carotid artery to block the origin of the. MCA. The animals were initially anaesthetised with 5% halothane in a 30% O₂: 70% N₂O mix. After intubation, the animals were mechanically ventilated and the level of halothane dropped to 1-1.5% to maintain anaesthesia. Body temperature was monitored using a rectal probe and maintained (36.8-37.2° C.) with the aid of a heating lamp. The left femoral artery was cannulated to allow for physiological monitoring in both the anaesthetised and conscious states. Rats were maintained normotensive (mean arterial blood pressure (MABP)≈80 mmHg), normocapnic (36<PaCO₂<44 mmHg) and adequately oxygenated (PO₂>100 mmHg) while under anaesthetic. Laser-Doppler Flowmetry (LDF, Moor Instrument Ltd.) was used to monitor cerebral blood flow, to confirm adequate ischaemia and subsequent reperfusion. If local cerebral blood flow was not immediately reduced with stabilization at less than 35% of the baseline signal, MCA occlusion was regarded as incomplete, and the animal was excluded from the study. The 35% threshold was based on the results of a pilot study for reproducibility in infarct size (data not shown).

Diathermy Occlusion of the MCA

The fur over the zygomatic arch was shaved and a 1 cm incision was made at right angles and rostral to the arch. The temporalis muscle was retracted and a fine probe inserted into the muscle to give an index of brain temperature during MCA occlusion (Aronowski et al., 1994). When mean arterial blood pressure (MABP) had stabilised a craniotomy was performed. The left middle cerebral artery (MCA) was exposed using a modification of the method described by Tamura et al. (1981). Briefly, through a 2 cm skin incision, the temporalis muscle was incised and stripped sub-periosteally from the lateral and ventral aspects of the temporal bone to enter the infratemporal fossa from the foramen opticum rostrally to the foramen ovale caudally. A small subtemporal craniectomy was made, centred 3 mm rostral to the foramen ovale, and the dura opened by a linear incision using a 25-gauge needle. Cerebral ischaemia was then induced by electrocoagulation of the MCA from a point proximal to the origin of the lenticulostriate artery.to a distal point where it crosses the inferior cerebral vein. The MCA was then transected at the olfactory tract to ensure completeness of the occlusion. The time of transection was taken as the exact time of MCA occlusion. The craniectomy wound was then sutured and the animal was allowed to recover from the anaesthesia. A subcutaneous injection of 2 ml of saline was given to prevent post-anaesthetic dehydration. After recovery from halothane the animal was monitored until its ventilation, heart rate and MABP had stabilised. The arterial cannula was then filled with a viscous heparinised solution (50% polyvinylpyrrolidone, 200 units heparin/ml) to maintain cannula patency, and the animal allowed to recover overnight, with soft food and water.

Intraluminal Thread Induced Ischaemia

Focal cerebral ischaemia was accomplished using a modification of the intraluminal thread model (4-0 nylon monofilament suture), first introduced by Koizumi et al. (1986). Briefly,. the right common, internal, and external carotid arteries were exposed through a ventral midline neck incision. The external carotid artery was ligated and then cut just proximal to the external carotid bifurcation. The common carotid artery was temporarily occluded with a microvascular clip. A 4-0 nylon monofilament was carefully inserted 20-23 mm from the bifurcation of the right common carotid artery, into the internal carotid artery via the external carotid artery, and advanced to block the origin of right MCA. At this point, the filament was either left in place, to produce maintained ischaemia within the MCA territory, or withdrawn after a defined period of time (e.g. 2 hours) to allow a period of reperfusion. The wound was then sutured and the animal allowed to recover from anaesthesia in the same way as for diathermy occlusion.

For both ischaemia models, experiments were terminated and the animal killed by perfusion fixation at defined time periods after the ischaemic insult. In most cases this was 24 hours after the onset of ischaemia.

Tissue Processing and Histological Quantification of Ischaemic Damage

The rats were perfusion fixed for neuropathological and immunohistochemical analysis with PAM (4% paraformaldehyde in PBS). Briefly, the rats were deeply anaesthetised with 5% halothane and placed in a supine position so that the thorax could be opened through a bilateral incision. A catheter was inserted into the left ventricle, the right atrium was incised, and heparinised saline was infused at a pressure equal to the MABP (90-110 mmHg) of the animal until the perfusate from the right atrium was bloodless. The saline was followed by approximately 300 ml of PAM. The rat was decapitated immediately after perfusion fixation, and the head stored in the fixative for at least 24 hours. The brain was then removed. After detaching the hindbrain, the forebrain was either cryoprotected in sucrose and then frozen (to generate fixed frozen sections) or processed in an automatic processor through alcohols and xylene and then embedded in paraffin wax; 70% ethanol for 2 hours; 80% ethanol for 3 hours; 96% ethanol for 4 hours; 4 separate stages in absolute ethanol for 4, 5, 5 and 6 hours; xylene/absolute ethanol (50:50) for 4 hours; 2 stages in xylene for 5 hours each. These 10 stages were carried out at 35° C. and were followed by 3 stages of 5, 5 and 6 hours in paraffin wax at 60° C. Paraffin sections of 5-7 μm were then cut on a microtome at multiple coronal levels for histology and immunohistochemistry. Frozen sections of 20-30 mm were similarly cut on a cryostat. Histology sections were collected on glass slides and stained with haematoxylin and eosin (H&E). Areas of ischaemic damage (Brierly and Graham, 1984) were identified on H&E sections by light microscopy and delineated at eight pre-selected coronal levels throughout the MCA territory (from anterior 10.50 mm to anterior 1.02 mm, Osborne et al. 1987). The areas of brain damage were drawn on scale diagrams (×3.36 actual size) of forebrain based in the atlas of Konig and Klippel (1963) and measured on an image analyser (MCID, M4, Imaging Research, St Catherine's, Ontario). These areas were then integrated, with the known distance between each coronal level, to determine the total volume of ischaemic damage in each specimen.

Double Label Immunofluorescence

After removal of the wax in Histoclear, the sections were dehydrated in absolute alcohol for 20 min and then microwaved for 10 min in 10 mM citric acid (pH 6.0) and allowed to cool at room temperature for 60 min. The sections were incubated with blocking solution of 50 mM phosphate buffered saline (PBS, pH 7.2) containing 0.5% bovine serum albumin and 10% normal horse serum. The mouse monoclonal antibody against PCNA (Santa Cruz; PC10) was diluted 1:1,000 in PBS, applied to sections and incubated overnight at 4° C. The sections were washed in PBS 2×20 min. Secondary antibody (Texas Red anti-mouse, 1:100, Vector Laboratories) was applied for one hour and the sections washed again (2×20 min). The washing procedure with PBS was repeated and then the sections were incubated with blocking solution of 50 mM phosphate buffered saline (PBS, pH 7.2) containing 0.5% bovine serum albumin and 10% normal goat serum. The polyclonal antiserum against GADD34 (Santa Cruz) was diluted 1:100 in PBS and was applied to sections and incubated overnight at 4° C. The sections were washed in PBS 2×20 min. Secondary antibody (fluoresceine conjugated anti-rabbit serum, 1:100, Vector Laboratories) was applied for one hour and the sections washed again 2×20 min. After washing the sections in PBS and water, the sections were mounted in immersion solution (Vector).

The following antibodies were used for double label immunofluorescence to classify cell types containing GADD34 or PCNA : neurones—Neu-N (mouse monoclonal Ab, Chemicon), astrocytes—GFAP (mouse monoclonal Ab, Sigma), microglia—Mrf-1 (rabbit polyclonal Ab, gift from Dr. S Tanaka, University of Hokkaido, Japan), following the same protocol as above.

Generation of the Polyclonal Antibodies Directed Against GADD34.

Oligonucleotides composed of 108 bases:

GADD34 1.
5′AATTCCATGGGACCAGCCCAGGCTGCCCGACGTGGTCCCTGGGAGCAG
CTTGCACGAGATAGGAGCCGTTTTGCTCGAAGAATTGCCCAGGCGGAGGA
GAAGCTGTAG 3′
and
GADD34 2
5′TCGACTACAGCTTCTCCTCCGCCTGGGCAATTCTTCGAGCAAAACGGC
TCCTATCTCGTGCAAGCTGCTCCCAGGGACCACGTCGGGCAGCCTGGGCT
GGTCCCATGG 3′

with an EcoRI site at one end and a SalI site at the other end were annealed and ligated with EcoRI/SalI cut pGEM 3ZF. The plasmid was transformed into NM522 E.coli, the DNA extracted and cut with EcoRI and SalI. The insert was separated on an agarose gel and purified by Spinnex tube centrifugation. The insert was then ligated into EcoRI/SalI cut pGEX 4T-3 and BL21 Epicurean E.coli were transformed. The bacterial culture was grown for 3 hr at 37° C. and induced for 2 hr at 37° C. with IPTG (1 μl /ml of 100 mM solution). The bacteria were spun down, resuspended in PBS, probe sonicated and the debris spun out. The supernatant extract was mixed with glutathione agarose beads for 40 min at room temperature. The beads were spun down and washed 3 times with PBS. The GST/GADD34 fusion protein was eluted with reduced glutathione elution buffer (Pharmacia). Several extractions were carried out and the protein content of each quantitated.

Four New Zealand white rabbits were each injected with six 0.5 ml (˜1 mg protein) inocula at 2 weekly intervals. The rabbits were bled out after the 5th boost and the sera analysed for specificity to GADD34 by Western blotting against BHK cell extracts. Four antisera were identified. These were designated 159, 160, 161 and 162.

Immnunohistochemistry

Multiple sections at 8 pre-selected coronal levels were processed for immunohistochemistry. Sections were mounted on Poly-L-Lysine (SIGMA) coated slides, dried at 37° C. overnight and then placed in Histoclear for 20 mins to remove the wax followed by dehydration in absolute alcohol for 20 min. They were then microwaved for 10 min in 10 mM citric acid (pH 6.0), allowed to cool at room temperature for 60 min, and incubated in 3% H₂O₂ in methanol for 30 min followed by one hour in 50 mM phosphate buffered saline (PBS, pH 7.2) containing 0.5% w bovine serum albumin and 10% normal goat or normal horse serum. A rabbit polyclonal antiserum against GADD34 (Brown et al., 1997) and mouse monoclonal antibody against PCNA (Santa Cruz; PC10) were diluted 1:1000 and 1:20,000 in PBS, respectively, and applied to the sections and incubated overnight at 4° C. The sections were washed in PBS 2×20 min. A secondary antibody (biotinylated goat anti-rabbit and horse anti-mouse, 1:100, Vector Laboratories) was applied for one hour and the sections washed again 2×20 min. The avidin/biotinylated horseradish peroxidase complex (ABC kit, Vector Laboratories) was applied for one hour. The washing procedure with PBS was repeated and then the sections allowed to develop in 3,3′-diaminobenzidine solution (Vector Laboratories) for 3 min for GADD34 and SG solution (Vector Laboratories) for 3 min for PCNA. Finally, the sections were dehydrated, cleared, and mounted for light microscopy analysis. Negative controls for GADD34 and PCNA antibodies, where the primary antibody was omitted from the procedure, were included in the protocol, and minimal staining was detected. All further analysis was performed “blind” by an investigator unaware of the identity of each section.

Immunopositive cells for GADD34 and PCNA were counted in microscopic fields (100 mm² grid at ×200 magnification) within the ischaemic core and peri-infarct zone at the coronal level of the caudate nucleus.

### Results

Expression of GADD34 and PCNA in Non-Ischaemic Tissue

In sham operated animals and in the MCA territory of the hemisphere contralateral. to the ischaemic insult, there was no evidence of GADD34 or PCNA immunopositive cell staining at 2 hours after MCA occlusion (FIG. 1, top right). However, cells immunopositive for PCNA were consistently observed in ependyma lining the ventricles bilaterally (FIG. 1, bottom right). In double label studies, these cells were found to co-express GADD34 (see FIG. 6, top right)

Expression of GADD34 and PCNA After Focal Ischaemia

Ischaemic damage was consistently identified in the ipsilateral MCA territory including the frontal cortex, the dorsal parietal cortex, and the caudate putamen with damage expanding over time into the frontoparietal cortex. Neuronal perikarya exhibited the characteristic morphological features of ischaemic damage, i.e. shrinkage and triangulation of the nucleus and cytoplasm, and increased eosinophilia of cytoplasm. The sharp transition between normal neuronal perikarya and ischaemic cell change was identifiable and transcribed onto scale diagrams.

i. Two Hours After Focal Cerebral Ischaemia

In animals sacrificed 2 hours after MCA occlusion, ischaemic damage was localised to the ipsilateral cerebral cortex and caudate putamen (FIG. 1, bottom right) with a total volume of damage of 48.4±8.3 mm³ (FIG. 2).

GADD34 immunopositive cells were present in the ipsilateral cortex and the caudate nucleus of all animals. The cells, which displayed a neuronal morphology and were predominantly distributed in the ipsilateral cortical neuronal layers (particularly those adjacent to the pial surface) of the peri-infarct zone (FIG. 1, top left). GADD34 immunopositive cells were also present in the irreversibly damaged ischaemic core though at a lower frequency than in the peri-infarct zone (FIG. 1, bottom right, FIG. 3, top left).

PCNA immunopositive cells were not detected consistently in either the ischaemic core or the peri-infarct zone at this time point (FIG. 3, bottom left).

ii. Twenty-Four Hours After Focal Cerebral Ischaemia

In animals sacrificed 24 hours after MCA occlusion, ischemic damage was localised to the ipsilateral cerebral cortex and caudate putamen with a total volume of 154±13.2 mm³ (FIG. 2).

GADD34 immuno-positive cells were predominantly distributed in the ipsilateral cortical neuronal layers of the peri-infarct zone (particularly near the pial surface) and most cells displayed a neuronal morphology (FIG. 4, top left). A small number of GADD34 immunopositive cells were also present in the irreversibly damaged ischaemic core (FIG. 4, top right and bottom right).

In the peri-infarct zone, GADD 34 predominantly co-localised with Neu-N, confirming neuronal expression of the protein (FIG. 5). Confocal microscopy revealed predoninantly cytoplasmic localisation of GADD34 with nuclear staining in some cells (FIG. 6 top left, FIG. 7 bottom row, middle panel). GADD34 did co-localise with Mrf-1 but did not co-localise with GFAP (FIG. 5, bottom panels) confirming that some microglia express GADD34 but astrocytes do not express the protein at this time-point. Double-label immunofluorescence revealed that GADD34 and PCNA immunoreactivity co-localized in some cells with a neuronal morphology in the peri-infarct zone (FIG. 6, top left). PCNA localised to the nucleus and there was cytoplasmic/nuclear GADD34 staining (FIG. 7, bottom row). Co-localisation of GADD34 and PCNA was also present in ependymal cells in the subventricular zone (FIG. 6, top right).

PCNA immunopositive cells were more extensively distributed by 24 hours compared to 2 hours after MCAO (FIG. 3, bottom right and FIG. 4, bottom panels). Most of the PCNA positive cells were distributed in the ipsilateral peri-infarct region including the cortex, corpus callosum and the caudate putamen (FIG. 4, bottom left). As described above, some cells with a neuronal morphology were PCNA positive but the majority of PCNA positive cells were microglia, (i.e. Mrf-1 positive, FIG. 6, bottom left, FIG. 7, top panels). PCNA-positive microglia were also observed in the cingulate cortex of both ipsilateral and contralateral hemispheres (FIG. 4, bottom right). Astrocytes in the peri-infarct region did not express PCNA (FIG. 6, bottom right).

Table 1 below provides a summary of the cellular localisation of GADD 34 and PCNA immunoreactivity at 24 hours after MCA occlusion.

TABLE 1
GADD34 and PCNA immunopositive cells 24 hours post-ischaemia. (−)
immunopositive cells not detected; (+,++) increasing numbers
of immunopositive cell; (nd) not determined.

| | GADD34 + | GADD34 + | GADD34 + | PCNA + | PCNA + | PCNA + | GADD34 + |
|---|---|---|---|---|---|---|---|
| Region | Neu-N | GFAP | Mrf−1 | Mrf−1 | GFAP | Neu-N | PCNA |
| Ischaemic Core | − | − | − | − | − | nd | − |
| Peri-infarct | ++ | − | + | ++ | − | nd | + |
| Zone | | | | | | | |
| Cingulate | + | − | − | + | − | nd | − |
| Cortex | | | | | | | |
| Subventricular | − | − | − | − | − | nd | ++ |
| zone | | | | | | | |

### Discussion

GADD34 and PCNA protein expression have. been characterised in a rat model of focal cerebral ischaemia. Two hours after MCA occlusion, significant numbers of GADD34 immunopositive cells were observed in the peri-infarct zone (penumbra) surrounding the core of ischaemic damage, with scattered immunopositive cells within the core region. Immediately following the ischaemic insult, protein synthesis in the brain is markedly suppressed but specific stress induced proteins (e.g. heat-shock proteins) are known to be expressed (Kogure and Kato, 1993; Sharp 2000). This up-regulation of specific gene expression may be integral to cell survival, repair or cell death. GADD34 is produced in response to ischaemic stress and its presence, specifically in cells at the boundary zone between irreversible and potentially reversible damage, points to an important role in influencing the fate of these cells. Evidence of GADD34 protein synthesis during the first few hours of ischaemia is compatible with a previous study, reporting transcription of GADD34 MRNA in response to global cerebral ischaemia (Doutehil et al., 1999).

Since the boundary between histologically normal and irreversibly damaged tissue is still advancing at 2 hours post insult, most of the cells in the peri-infarct zone are in the process of dying, with many becoming irreversibly damaged by 24 hours. The expression of GADD34 in cells within this zone indicates its involvement either in the processes of cell death or survival. By 24 hours post-ischaemia, increased numbers of GADD34 positive cells, most of which are neurons, are present in the peri-infarct zone. In some of these cells GADD34 colocalises with PCNA. The increase in GADD34 positive cells coupled to its colocalisation with PCNA suggests that, at this stage, GADD34 is functioning in the process of DNA repair and replication to limit further tissue loss. The present inventors have shown previously that the 63 amino acid domain, conserved in GADD34 and HSV ICP34.5, specifically complexes with PCNA in vitro (Brown et al., 1997). The available data therefore suggest that either independently or in interaction with PCNA, GADD34 plays a role in cell survival by allowing DNA replication to continue.

As well as being present in neurons in the peri-infarct zone, PCNA was also observed in the adjacent cingulate cortex in the ipsilateral hemisphere and in the contralateral cingulate cortex. PCNA is normally synthesized during the S-phase in the cell cycle, although it is also present at very low levels in quiescent cells (Miyachi et al., 1978). Double label immunofluorescence with microglial response factor-1 indicated that most PCNA immunoreactive cells in the peri-infarct zone, in the cingulate cortex of the ipsilateral hemisphere and in the cingulate cortex of the contralateral hemisphere were microglia. GFAP positive astrocytes did not show PCNA positive staining. This result is in keeping with a report by Norton (1999) where it was shown that microglia were the first cells. to divide following brain injury and constituted the bulk of dividing cells.

During focal ischaemia, protein synthesis can be completely suppressed since the eukaryotic translation initiation factor 2α (eIF2α) is phosphorylated by protein kinase R which is sensitive to decreased oxygen and increases of adenosine monophosphate (Srivastava et al., 1998). Ischaemia-induced phosphorylation of eIF2α is believed to be a major cause of the inhibition of protein synthesis and subsequent induction of apoptosis in vulnerable neurons (DeGracia 1997). Dephosphorylation of eIF2α is in turn a crucial step for the return to normal protein synthesis and protection of injured neurons (Sullivan et al., 1999). The C-terminal 63 amino acids of GADD34 has been shown to interact with phosphatase 1α (PP1α) to actively dephosphorylate the eukaryotic translation initiation factor 2α (eIF2α) subunit in order to preclude protein synthesis inhibition and inhibit stress-induced gene expression (Sheikh L Fornace 1999; Novoa et al., 2001). GADD34 expression in a high proportion of cells in the peri-infarct region suggests that the protein is being synthesised to dephosphorylate eIF2α and therefore to preclude protein synthesis. inhibition.

In summary, evidence is provided that GADD34 and PCNA are up-regulated in the peri-infact zone in response to an ischaemic insult. In the acute stage of ischaemia, it appears that GADD34 is implicated in the control of cell death or survival. At later times after the induction of ischaemia, the present results suggest that GADD34, per se, or in interaction with PCNA, plays a role in cell survival by promoting DNA replication and essential protein expression.

### References

Aronowski J, Ostrow P, Samways E, Strong R, Zivin J A, Grotta J C (1994) Graded bioassay for demonstration of brain rescue from experimental acute ischaemia in rats. Stroke 25, 2235-2240.

Brierley J B, Graham D I (1984) Hypoxia and vascular disorders of the central nervous system. In: Greenfield's Neuropathology, Adams J H, Corsellis JAN, Duchen L W (eds.), 4th Edition, pp125-207, Edward Arnold: London Brown S M, MacLean A R, McKie E A, Harland J (1997) The herpes simplex virus virulence factor ICP34.5 and the cellular protein MyD116 complex with proliferating cell nuclear antigen through the 63-amino-acid domain conserved in ICP34.5, MyD116, and GADD34. J Virol 71: 9442-9

Chou J, Roizman B (1992) The gamma 1 (34.5) gene of herpes simplex virus 1 precludes neuroblastoma cells from triggering total shutoff of protein synthesis characteristic of programed cell death in neuronal cells. Proc Natl Acad Sci USA 89: 3266-70

Chou J, Roizman B (1994) Herpes simplex virus 1 gl 34.5 gene function, which blocks the host response to infection, maps in the homologous domain of the genes expressed during growth arrest and DNA damage. Proc Natl Acad Sci USA 91: 5247-5251

DeGracia D J, Sullivan J M, Neumar R W, Alousi S S, Hikade K R,

Pittman J E, White B C, Rafols J A, Krause G S (1997) Effect of brain ischemia and reperfusion on the localization of phosphorylated eukaryotic initiation factor 2 alpha. J Cereb Blood Flow Metab 17:1291-302

Doutheil J, Althausen S, Gissel C, Paschen W (1999) Activation of MYD116 (gadd34) expression following transient forebrain ischemia of rat: implications for a role of disturbances of endoplasmic reticulum calcium homeostasis. Brain Res Mol Brain Res 63: 225-32

Fornace A J Jr, Nebert D W, Hollander M C, Luethy J D, Papathanasiou M, Pargnoli J, Holbrook N J (1989) Mammalian genes coordinately regulated by growth arrest signals and DNA-damaging agents. Mol Cell Biol 9: 4196-203

He B, Gross M, Roizman B (1997) The gamma(1)34.5 protein of herpes simplex virus 1 complexes with protein phosphatase lalpha to dephosphorylate the alpha subunit of the eukaryotic translation initiation factor 2 and preclude the shutoff of protein synthesis by double-stranded RNA-activated protein kinase. Proc Natl Acad Sci USA 94: 843-8.

Kogure K, Kato H (1993) Altered gene expression in cerebral ischemia. Stroke 24: 2121-2127

Konig J F R and R A Klippel. The rat brain. A stereotaxic atlas of the forebrain and lower parts of the brain stem. Kreiger. N.Y., 1963.

Lord K A, Hoffman-Liebermann B, Liebermann D A (1990) Sequence of MyD116 cDNA: a novel myeloid differentiation primary response gene induced by IL6. Nucleic Acids Res 18:2823

McGeoch, DJ, BC Barnett (1991) Neurovirulence factor. Nature 353: 609

Miyachi K, Fritzler M J, Tan E M (1978) Autoantibody to a nuclear antigen in proliferating cells. J Immunol 121: 2228-34

Norton W T (1999) Cell reactions following acute brain injury: a review. Neurochem Res 24: 213-8

Novoa I, Zeng H, Harding H P, Ron D (2001) Feedback inhibition of the unfolded protein response by GADD34-mediated dephosphorylation of eIF2a. The J of Cell Biol 153:1011-1021

Osborne K A, Shigeno T, Balarsky A M, Ford I, McCulloch J, Teasdale G M, Graham D I (1987) Quantitative assessment of early brain damage in a rat model of focal cerebral ischaemia. J Neurol Neurosurg Psychiatry 50:402-410.

Prelich G, Kostura M, Marshak D R, Mathews M B, Stillman B (1987) The cell-cycle regulated proliferating cell nuclear antigen is required for SV40 DNA replication in vitro. Nature 326: 471-5

Sharp F R, Lu A, Tang Y, Millhorn D E (2000) Multiple molecular penumbras after focal cerebral ischemia. J Cereb Blood Flow Metab 20:1011-32

Sheikh M S, Fornace A J Jr (1999) Regulation of translation initiation following stress. Oncogene 18: 6121-8

Srivastava S P, Kumar K U, Kaufman R J (1998) Phosphorylation of eukaryotic translation initiation factor 2 mediates apoptosis in response to activation of the double-stranded. RNA-dependent protein kinase. J Biol Chem 273: 2416-23

Sullivan J M, Alousi S S, Hikade K R, Bahu N J, Rafols J A, Krause G S, White B C (1999) Insulin induces dephosphorylation of eukaryotic initiation factor 2alpha and restores protein synthesis in vulnerable hippocampal neurons after transient brain ischemia. J Cereb Blood Flow Metab 19:1010-9

Tamura A, D I Graham, J McCulloch and G M Teasdale (1981) Focal cerebral ischaemia in the rat: description of technique and early neuropathological consequences following middle cerebral artery occlusion. J. Cereb. Blood Flow Metab. 1, 53-60.

1. Use of a GADD34 polypeptide, or a homologue or a derivative thereof, in the preparation of a medicament for the treatment of focal cerebral ischaemic damage.

2. Use of a polypeptide according to claim 1, wherein the medicament comprises a pharmaceutically active compound derived from GADD34.

3. Use of a polypeptide according to claim 1 or wherein the medicament comprises a viral vector designed to introduce a polynucleotide fragment encoding such a polypeptide into a brain cell.

4. Use of a polypeptide according to claim 3 wherein the polynucleotide fragment further comprises a hypoxic response element (HRE) promoter.

5. Use of a PCNA polypeptide, or a homologue or a derivative thereof, in the preparation of a medicament for the treatment of focal cerebral ischaemic damage.

6. Use of a polypeptide according to claim 5, wherein the medicament comprises a pharmaceutically active compound derived from PCNA.

7. Use of a polypeptide according to claim 5 or 6 wherein the medicament comprises a viral vector designed to introduce a polynucleotide fragment encoding such a polypeptide into a brain cell.

8. Use of a polypeptide according to claim 7 wherein the polynucleotide fragment further comprises a hypoxic response element (HRE) promoter.

9. A method of treatment of focal cerebral ischaemic damage, the method comprising the step of administering a pharmacologically active dose of a GADD34 polypeptide, or a homologue or a derivative thereof, to a human or animal patient in need of such treatment.

10. A method of treatment of focal cerebral ischaemic damage, the method comprising the step of administering a pharmacologically active dose of a PCNA polypeptide, or a homologue or a derivative thereof, to a human or animal patient in need of such treatment.

11. Use of an antibody to a GADD34 polypeptide for the delivery of a drug for treatment of focal cerebral ischaemic damage.

12. A method of diagnosis of focal cerebral ischaemic damage, the method comprising the steps of administering antibodies to a GADD34 polypeptide to a patient; and detecting the region of localisation of said antibodies.

13. The method of claim 14 wherein the antibodies are labelled to assist detection.

14. A pharmaceutical formulation comprising GADD34 or a physiologically acceptable salt, ester or other physiologically functional derivative thereof and a carrier therefor for use in treatment of focal cerebral ischaemia in a human or animal.

15. A pharmaceutical formulation comprising PCNA or a physiologically acceptable salt, ester or other physiologically functional derivative thereof and a carrier therefor for use in treatment of focal cerebral ischaemia in a human or animal.
